(19) United States
(12) Patent Application Publication
(10) Pub. No.: US 2011/0002877 A1
(43) Pub. Date: Jan. 6, 2011

(54) PTEN Inhibitors Treat Neutropenia-Associated Pneumonia

(76) Inventors: Hongbo R. Luo, Boston, MA (US); Yitang Li, Boston, MA (US)

Correspondence Address: RICHARD ARON OSMAN, 3525 Del Mar Heights Rd. #915, San Diego, CA, 92130, US

(21) Appl. No.: 12/497,629

(22) Filed: Jul. 3, 2009

(51) Int. Cl. A61K 31/785; A61K 38/18; A61P 31/04; A61K 31/34; A61K 31/4192; A61K 31/4164

(52) U.S. Cl. 424/78.37; 514/474; 514/359; 514/396; 424/85.1; 514/2.3

(57) ABSTRACT

Neutropenia-associated pneumonia is treated with a therapeutically effective amount of a small-molecule PTEN inhibitor. The method may further include detecting a resultant alleviation of the neutropenia-associated pneumonia, and/or diagnosing the neutropenia-associated pneumonia. The patient may be undergoing anti-cancer chemotherapy and/or radiotherapy, and the methods may further include the step of treating the patient with broad-spectrum antibiotic therapy or granulocyte colony-stimulating factor (G-CSF) therapy.

This work was supported by NIH grants HL085100 and GM076084. The U.S. government has certain rights in any patent issuing on this application.

### INTRODUCTION

The field of the invention is using PTEN inhibitors to treat neutropenia-associated pneumonia.

Pneumonia is usually triggered when a person's defense system is weakened¹⁻⁴. It represents a major cause of infectious complication in cancer patients with treatment-related neutropenia⁵⁻⁷. Neutropenia-related lung infections have been treated with broad-spectrum antibiotic therapy and granulocyte colony-stimulating factor (G-CSF) therapy. Here, we explored an alternative strategy for augmenting host defense in neutropenia-related pneumonia by enhancing neutrophil functions (i.e., recruitment, survival, and bacteria killing) in neutropenic patients. We achieved this by augmenting the intracellular PtdIns(3,4,5)P3 signaling pathway, which has been implicated in a variety of neutrophil functions, such as survival, polarization, chemotaxis, and NADPH oxidase activation⁸⁻¹¹. We recently demonstrated that augmenting PtdIns(3,4,5)P3 signal by depleting PTEN, a phosphatidylinositol 3′-phosphatase that negatively regulates PtdIns(3,4,5)P3 signaling, prevents neutrophil death¹². In addition, PTEN-null neutrophils had enhanced sensitivity to chemoattractant stimulation. A larger fraction of these neutrophils displayed membrane ruffles in response to chemoattractant stimulation. Chemoattractant-induced transwell migration and superoxide production were also augmented¹³. Here we show that PTEN disruption and inhibition in neutropenia-related pneumonias can alleviate pneumonia-associated lung damage and decrease the related mortality rate.

Augmenting neutrophil function by elevating the PtdIns(3,4,5)P3 signaling can increase aggravated inflammation and tissue damage. For example, in PTEN knockout mice we observed more severe pulmonary edema and increased mortality rate when bacterial pneumonia was induced in non-neutropenic mice. Furthermore, Heit et al. reported that PTEN is required for prioritizing and integrating responses to multiple chemotactic cues, and disruption of PTEN leads to ‘distraction’ in migrating neutrophils in an in vivo model of inflammatory arthritis⁵⁶. Despite these contraindications, we show that we can successfully use small-molecule PTEN inhibitors to treat patients with neutropenia-associated pneumonia, wherein the number of neutrophils is reduced and the release of noxious compounds, such as oxidants, proteinases, and DNA, by neutrophils is therapeutically tolerable.

### SUMMARY OF THE INVENTION

The invention provides methods and compositions for treating a patient for neutropenia-associated pneumonia. The general method comprises the step of: (a) administering to the patient a therapeutically effective amount of a small-molecule PTEN inhibitor.

In particular embodiments, the PTEN inhibitor is: (I) an ascorbic acid-based PTEN inhibitor; (II) a 1,2,3-triazole PTEN inhibitor; (III) a diamide PTEN inhibitor; (IV) an aryl imidazole carbonyl PTEN inhibitor; (V) a polyamide PTEN inhibitor; (VI) a selected commercial PTEN inhibitor; (VII) a 1,10-phenanthroline-5,6-dione PTEN inhibitor; (VIII) a substituted phenathrene-9-10-dione PTEN inhibitor; (IX) an Isatin (1H-indole-2,3-dione) PTEN inhibitor; (X) a substituted phenanthren-9-ol PTEN inhibitor; (XI) a substituted naphthalene-1,2-dione PTEN inhibitor; (XII) a substituted naphthalene-1,4-dione PTEN inhibitor; (XIII) a vanadate-based PTEN inhibitor; and (XIV) a T1-loop binding element containing PTEN inhibitor; including tautomers, stereoisomers and pharmaceutically-acceptable salts thereof.

The selected commercial PTEN inhibitors (VI) are (a) Deltamethrin; (S)-a-cyano-3-phenoxybenzyl(1R)-cis-3-(2,2 dibromovinyl)-2,2-dimethylcyclopropanecarboxylate; (b) Alendronate, sodium, trihydrate; (c) N-(9,10-Dioxo-9,10-dihydrophenanthren-2-yl)-2,2-dimethylpropionamide; (d) 5-benzyl-3furylmethyl (1R,S)-cis,trans-chrysanthemate; (e) Suramin, Sodium Salt; 8,8′-[carbonylbis[imino-3, lphenylenecarbonylimino (4-methyl-3,1-phenylene)carbonylimino]]bis-, hexasodium salt; (f) 4-methoxyphenacyl bromide; (g) 1,4-dimethylendothall; 1,4-dimethyl-7-oxabicyclo[2.2.1]heptane-2,3-dicarboxylic acid; (h) Cantharidic acid; 2,3-dimethyl-7-oxabicyclo[2.2.1]heptane-2,3dicarboxylic acid; (i) Sodium Stibogluconate; antimony sodium gluconate; (j) 3,4-Dephostatin, ethyl-; (k) Fenvalerate; α-cyano-3-phenoxybenzyl-α-(4-chlorophenyl)isovalerate; (l) α-naphthyl acid phosphate, monosodium salt; (m) β-glycerophosphate, disodium salt, pentahydrate; (n) Endothall; 7-oxabicyclo[2.2.1]heptane-2,3dicarboxylic acid; and (o) Cypermethrin; (R,S)-α-cyano-3-phenoxybenzyl-3 (2,2-dichlorovinyl)-2,2-dimethylcyclopropanecarboxylate; (1R)-(R)cyano(3-phenoxyphenyl)methyl 3-(2,2-dichlorovinyl)-2,2-dimethylcyclopropanecarboxylate.

In particular embodiments, the method further comprises the step of detecting a resultant alleviation of the neutropenia-associated pneumonia, and/or the step of diagnosing the neutropenia-associated pneumonia.

In particular embodiments, the patient is undergoing or is to undergo anti-cancer chemotherapy and/or radiotherapy. The methods may further comprise the step of treating the patient with broad-spectrum antibiotic therapy or granulocyte colony-stimulating factor (G-CSF) therapy.

In another aspect, the invention provides compositions specifically adapted to the subject methods, such as copackaged or coformulated compositions comprising, preferably in unit dosages, therapeutically-effective amounts of a PTEN inhibitor and/or a broad-spectrum antibiotic or granulocyte colony-stimulating factor (G-CSF).

### DETAILED DESCRIPTION OF SPECIFIC EMBODIMENTS OF THE INVENTION

In one embodiment, the invention provides methods of treating a patient in need thereof for neutropenia-associated pneumonia, comprising the step of: (a) administering to the patient a therapeutically effective amount of a small-molecule PTEN inhibitor. Target patients are determined to have or be specifically predisposed neutropenia-associated pneumonia, and include patients diagnosed with neutropenia-associated pneumonia and patients exposed or expected to be exposed to conditions known to cause or contribute to neutropenia-associated pneumonia, such as patients undergoing or to undergo (e.g. scheduled to undergo) anti-cancer chemotherapy or radiotherapy which contributes to neutropenia-associated pneumonia or increased susceptibility thereto.

The method may be practiced in conjunction with additional steps or treatments applicable to the subject target patient, such as of co-treating a patient patients undergoing or to undergo anti-cancer chemotherapy or radiotherapy with broad-spectrum antibiotic therapy or granulocyte colony-stimulating factor (G-CSF) therapy.

Small molecule PTEN inhibitors can administered via a route of administration including, but not limited to, oral, i.v., sub-cutaneous, i.v. drip, intramuscular, nasally as aerosol, dermal patch, mucous exposure, etc as compatible conventional formulations or as drug delivery modalities such as slow release formulations, depots, liposomes, microparticles, nanoparticles, and degradable and/or targeted versions thereof, preferably orally-admininstrable/orally-active.

Depending on the intended route of delivery, the PTEN inhibitors may be administered in one or more dosage form(s), e.g. liquid, ointment, solution, suspension, emulsion, tablet, capsule, caplet, lozenge, powder, granules, cachets, douche, suppository, cream, mist, eye drops, gel, inhalant, patch, implant, injectable, infusion, etc. The dosage forms may include a variety of other ingredients, including binders, solvents, bulking agents, plasticizers etc.

Suitable PTEN inhibitors include PTEN inhibitor compounds of formulas I-XIV as described herein and in US2007/0203098 and WO2005/097119; vanadium-based PTEN inhibitors described in US20070292532 and by Rosivatz et al. 2006 (ACS Chem. Biol. 1(12) 780-790); the 1,4-naphthoquinone derivative, shikonin, described by Nigorikawa et al. (Mol Pharmacol 70:1143-1149, 2006); and menadione (vitamin K3) as described by Yoshikawa et al., Biochim Biophys Acta. 2007 April; 1770(4):687-93. PTEN inhibition assays for general screening (to identify and confirm alternative, suitable inhibitors) and IC50 determinations are described herein and/or known in the art, e.g. US2007/0203098, WO2005/097119, Example 11, below.

Particularly suitable PTEN inhibitors and applicable manufacturing methods are also described in US2007/0203098, including all recited genera, subgenera and species disclosed and as described therein including as follows.

As used herein, the singular forms “a,” “an” and “the” include plural referents unless the context clearly dictates otherwise.

The term “branched” as used herein refers to a group containing from 1 to 24 backbone atoms wherein the backbone chain of the group contains one or more subordinate branches from the main chain. Preferred branched groups herein contain from 1 to 12 backbone atoms. Examples of branched groups include, but are not limited to, isobutyl, t-butyl, isopropyl, —CH₂CH₂CH(CH₃)CH₂CH₃, —CH₂CH(CH₂CH₃)CH₂CH₃, —CH₂CH₂C(CH₃)₂CH₃, —CH₂CH₂C(CH₃)₃ and the like.

The term “unbranched” as used herein refers to a group containing from 1 to 24 backbone atoms wherein the backbone chain of the group extends in a direct line. Preferred unbranched groups herein contain from 1 to 12 backbone atoms.

The term “cyclic” or “cyclo” as used herein alone or in combination refers to a group having one or more closed rings, whether unsaturated or saturated, possessing rings of from 3 to 12 backbone atoms, preferably 3 to 7 backbone atoms.

The term “lower” as used herein refers to a group with 1 to 6 backbone atoms.

The term “saturated” as used herein refers to a group where all available valence bonds of the backbone atoms are attached to other atoms. Representative examples of saturated groups include, but are not limited to, butyl, cyclohexyl, piperidine and the like.

The term “unsaturated” as used herein refers to a group where at least one available valence bond of two adjacent backbone atoms is not attached to other atoms. Representative examples of unsaturated groups include, but are not limited to, —CH₂CH₂CH═CH₂, phenyl, pyrrole and the like.

The term “aliphatic” as used herein refers to an unbranched, branched or cyclic hydrocarbon group, which may be substituted or unsubstituted, and which may be saturated or unsaturated, but which is not aromatic. The term aliphatic further includes aliphatic groups, which comprise oxygen, nitrogen, sulfur or phosphorous atoms replacing one or more carbons of the hydrocarbon backbone.

The term “aromatic” as used herein refers to an unsaturated cyclic hydrocarbon group having 4n+2 delocalized pi electrons, which may be substituted or unsubstituted.

The term aromatic further includes aromatic groups, which comprise a nitrogen, oxygen, or sulfur atom replacing one or more carbons of the hydrocarbon backbone. Examples of aromatic groups include, but are not limited to, phenyl, naphthyl, thienyl, furanyl, pyridinyl, (is)oxazoyl and the like.

The term “substituted” as used herein refers to a group having one or more hydrogens or other atoms removed from a carbon or suitable heteroatom and replaced with a further group. Preferred substituted groups herein are substituted with one to five, most preferably one to three substituents. An atom with two substituents is denoted with “di,” whereas an atom with more than two substituents is denoted by “poly.” Representative examples of such substituents include, but are not limited to aliphatic groups, aromatic groups, alkyl, alkenyl, alkynyl, aryl, alkoxy, halo, aryloxy, carbonyl, acryl, cyano, amino, nitro, phosphate-containing groups, sulfur-containing groups, hydroxyl, alkylcarbonyloxy, arylcarbonyloxy, alkoxycarbonyloxy, aryloxycarbonyloxy, alkylcarbonyl, arylcarbonyl, alkoxycarbonyl, aminocarbonyl, alkylaminocarbonyl, dialkylaminocarbonyl, alkylthiocarbonyl, acylamino, amidino, imino, alkylthio, arylthio, thiocarboxylate, alkylsulfinyl, trifluoromethyl, azido, heterocyclyl, alkylaryl, heteroaryl, semicarbazido, thiosemicarbazido, maleimido, oximino, imidate, cycloalkyl, cycloalkylcarbonyl, dialkylamino, arylcycloalkyl, arylcarbonyl, arylalkylcarbonyl, arylcycloalkylcarbonyl, arylphosphinyl, arylalkylphosphinyl, arylcycloalkylphosphinyl, arylphosphonyl, arylalkylphosphonyl, arylcycloalkylphosphonyl, arylsulfonyl, arylalkylsulfonyl, arylcycloalkylsulfonyl, CF₃, combinations thereof, and substitutions thereto.

The term “alkyl” as used herein alone or in combination refers to a branched or unbranched, saturated aliphatic group. Representative examples of alkyl groups include, but are not limited to, methyl, ethyl, n-propyl, isopropyl, n-butyl, isobutyl, sec-butyl, tert-butyl, octyl, decyl, tetradecyl, hexadecyl, eicosyl, tetracosyl and the like.

The term “alkenyl” as used herein alone or in combination refers to a branched or unbranched, unsaturated aliphatic group containing at least one carbon-carbon double bond which may occur at any stable point along the chain. Representative examples of alkenyl groups include, but are not limited to, ethenyl, E- and Z-pentenyl, decenyl and the like.

The term “alkynyl” as used herein alone or in combination refers to a branched or unbranched, unsaturated aliphatic group containing at least one carbon-carbon triple bond which may occur at any stable point along the chain. Representative examples of alkynyl groups include, but are not limited to, ethynyl, propynyl, propargyl, butynyl, hexynyl, decynyl and the like.

The term “aryl” as used herein alone or in combination refers to a substituted or unsubstituted aromatic group, which may be optionally fused to other aromatic or non-aromatic cyclic groups. Representative examples of aryl groups include, but are not limited to, phenyl, pyridyl-, furazan, benzyl, naphthyl, benzylidine, xylyl, styrene, styryl, phenethyl, phenylene, benzenetriyl and the like.

The term “alkoxy” as used herein alone or in combination refers to an alkyl, alkenyl or alkynyl group bound through a single terminal ether linkage. Examples of alkoxy groups include, but are not limited to, methoxy, ethoxy, n-propoxy, iso-propoxy, n-butoxy, 2-butoxy, tertbutoxy, n-pentoxy, 2-pentoxy, 3-pentoxy, isopentoxy, neopentoxy, n-hexoxy, 2-hexoxy, 3-hexoxy, 3-methylpentoxy, fluoromethoxy, difluoromethoxy, trifluoromethoxy, chloromethoxy, dichloromethoxy, and trichloromethoxy.

The term “aryloxy” as used herein alone or in combination refers to an aryl group bound through a single terminal ether linkage.

The term “halogen,” “halide” or “halo” as used herein alone or in combination refers to fluorine, chlorine, bromine, iodine, and astatine. Representative examples of halo groups include, but are not limited to, chloroacetamido, bromoacetamido, idoacetamido and the like.

The term “hetero” as used herein combination refers to a group that includes one or more atoms of any element other than carbon or hydrogen. Representative examples of hetero groups include, but are not limited to, those groups that contain heteroatoms including, but not limited to, nitrogen, oxygen, sulfur and phosphorus.

The term “heterocycle” as used herein refers to a cyclic group containing a heteroatom. Representative examples of heterocycles include, but are not limited to, pyridine, piperadine, pyrimidine, pyridazine, piperazine, pyrrole, pyrrolidinone, pyrrolidine, morpholine, thiomorpholine, indole, furazan, isoindole, imidazole, triazole, tetrazole, furan, benzofuran, dibenzofuran, thiophene, thiazole, benzothiazole, benzoxazole, benzothiophene, quinoline, isoquinoline, azapine, naphthopyran, furanobenzopyranone and the like.

The term “carbonyl” or “carboxy” as used herein alone or in combination refers to a group that contains a carbon-oxygen double bond. Representative examples of groups which contain a carbonyl include, but are not limited to, aldehydes (i.e., formyls), ketones (i.e., acyls), carboxylic acids (i.e., carboxyls), amides (i.e., amidos), imides (i.e., imidos), esters, anhydrides and the like.

The term “acryl” as used herein alone or in combination refers to a group represented by CH₂═C(Q)C(O)O— where Q is an aliphatic or aromatic group.

The term “cyano,” “cyanate,” or “cyanide” as used herein alone or in combination refers to a carbon-nitrogen double bond or carbon-nitrogen triple bond. Representative examples of cyano groups include, but are not limited to, isocyanate, isothiocyanate and the like.

The term “amino” as used herein alone or in combination refers to a group containing a backbone nitrogen atom. Representative examples of amino groups include, but are not limited to, alkylamino, dialkylamino, arylamino, diarylamino, alkylarylamino, alkylcarbonylamino, arylcarbonylamino, carbamoyl, ureido and the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thios, thioxos and the like.

The term “pharmaceutically acceptable salts” is meant to include salts of the active compounds which are prepared with relatively nontoxic acids or bases, depending on the particular substituents found on the compounds described herein. When the compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al. (1977) J. Pharm. Sci. 66:1-19). Some compounds of the invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides inhibitor compounds which are in a prodrug form. Prodrugs of the compounds described herein are those compounds that undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the invention which is administered as an ester (the “prodrug”), but then is metabolically hydrolyzed to the carboxylic acid, the active entity. Additional examples include peptidyl derivatives of a compound of the invention.

Certain compounds of the invention can exist in unsolvated forms as well as solvated forms, including hydrated forms. In general, the solvated forms are equivalent to unsolvated forms and are intended to be encompassed within the scope of the invention. Certain compounds of the invention may exist in multiple crystalline or amorphous forms. In general, all physical forms are equivalent for the uses contemplated by the invention and are intended to be within the scope of the invention.

Certain compounds of the invention possess asymmetric carbon atoms (optical centers) or double bonds; the racemates, diastereomers, geometric isomers and individual isomers are all intended to be encompassed within the scope of the invention.

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “therapeutically effective amount” refers to the amount of the subject compound that will elicit, to some significant extent, the biological or medical response of a tissue, system, animal or human that is being sought by the researcher, veterinarian, medical doctor or other clinician, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The subject PTEN inhibitors compounds include:

I) Ascorbic Acid-Based PTEN Inhibitors:

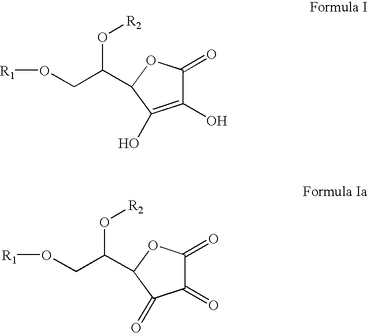

wherein,
R1 represents H, C1-C3 alkyl, aryl, alkylaryl, (CH₂)ₙCOXR3, (CH₂)ₙXCOR3, (CH₂)ₙCOR3, (CH₂)ₙSO₂R3, (CH₂)ₙXR3, (CH₂)ₙSO₂X—R3, (CH₂)ₙXSO₂R3, (CH₂)ₙNR3R4, or (CH₂)ₙCO(CH₂)ₘXR3;
R2 represents H, C1-C3 alkyl, aryl, alkylaryl, (CH₂)ₙCOX—R3, (CH₂)ₙXCOR3, (CH₂)ₙCOR3, (CH₂)ₙSO₂R3, (CH₂)ₙXR3, (CH₂)ₙSO₂X—R3, (CH₂)ₙXSO₂R3, (CH₂)ₙNR3R4, or (CH₂)ₙCO(CH₂)ₘXR3;
R3, R5 and R6 independently are H, C1-C4 alkyl, aryl or alkylaryl;
R4 represents H, C1-C4 alkyl, aryl, alkylaryl, NHSO₂R5, NHCO₂R5, or NR5R6;
m=0 to 3;
n=0 to 3; and
X represents O or NR₄.
Compounds of Formula I and Ia may have ester linkages at either R1 or R2.

II) 1,2,3-triazole PTEN Inhibitors (Such as Described in WO2002/32896):

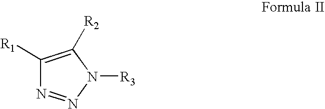

wherein,
R1 represents H, C1-C4 alkyl, aryl, alkylaryl, COXR2, COR2, SO₂XR2, SO₂R2;
R2 represents H, C1-C4 alkyl, aryl, alkylaryl, (CH₂)ₙCOXR4, (CH₂)ₙXCOR4, (CH₂)ₙXR4, (CH₂)ₙSO₂XR4, (CH₂)ₙXSO₂R4, NHSO₂R4, NHCOR4, NHCO₂R4, NHCOCO₂R4, or NR4R5;
R3 represents H, C1-C4 alkyl, aryl, alkylaryl, (CH₂)ₙCOXR4, (CH₂)ₙXCOR4, (CH₂)ₙXR4, (CH₂)ₙSO₂XR4, (CH₂)ₙXSO₂R4, NHSO₂R4, NHCOR4, NHCO₂R4, NHCOCO₂R4, or NR4R5;
R4 represents H, C1-C4 alkyl, aryl, or alkylaryl;
R5 represents H, C1-C4 alkyl, aryl, alkylaryl, NHSO₂R6, NHCOR6, NHCO₂R6, NR6R7, or N═C(R6R7);
R6 represents H, C1-C4 alkyl, aryl, or alkylaryl;
R7 represents H, C1-C4 alkyl, aryl, or alkylaryl;
n=0-3; and
X represents O or NR5.

The inhibitors of Formula II include:

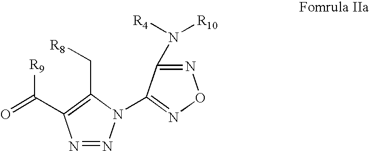

wherein,
R8 represents (CH₂)ₙXR4, or (CH₂)ₙSR4;
R9 represents NHNHSO₂-aryl, NHNHCO-aryl, or NHN═C(R6R7); and
R10 represents H, C1-C4 alkyl, aryl, alkylaryl, SO₂R6, COR6, or CO₂R6.

III) Diamide PTEN Inhibitors

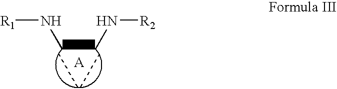

wherein,
A is a five or six member ring;
R1 represents H, C1-C3 alkyl, aryl, alkylaryl, (CH₂)ₙCOXR3, (CH₂)ₙXCOR3, (CH₂)ₙCOR3, (CH₂)ₙSO₂R3, (CH₂)ₙXR3, (CH₂)ₙSO₂XR3, (CH₂)ₙXSO₂R3, NHSO₂R3, NHCO₂R3, NHCOR3, NHCO₂R3, NHCOCO₂R3, NR3R4, or (CH₂)ₙCO(CH₂)ₘXR3;
R2 represents H, C1-C3 alkyl, aryl, alkylaryl, (CH₂)ₙCOXR3, (CH₂)ₙXCOR3, (CH₂)ₙCOR3, (CH₂)ₙSO₂R3, (CH₂)ₙXR3, (CH₂)ₙSO₂XR3, (CH₂)ₙXSO₂R3; NHSO₂R3, NHCO₂R3, NHCOR3, NHCO₂R3, NHCOCO₂R3, NR3R4, or (CH₂)ₙCO(CH₂)ₘXR3;
R3 represents H, C1-C4 alkyl, aryl, or alkylaryl;
R4 represents H, C1-C4 alkyl, aryl, alkylaryl, NHSO₂R5, NHCO₂R5, or NR5R6;
R5 represents H, C1-C4 alkyl, aryl, or alkylaryl;
R6 represents H, C1-C4 alkyl, aryl, or alkylaryl;
n=0-3;
m=0-3; and
X represents O, or NR4.

Ring A may be saturated, unsaturated, or aromatic, and may optionally comprise N and O. Preferred compounds of formula III are those wherein ring A is selected from heterocyclic ring systems, especially vicinally substituted pyridines, pyrimidines, furazans, imidazoles, pyrrazoles, furaus, thiazoles, and oxazoles, as well as their saturated analogs; other preferred inhibitors of formula III are those wherein ring A comprises an all carbon aromatic rings, such as substituted and unsubstituted phenyl, and their saturated analogs.

The inhibitors of Formula III may comprise a ring A selected from the following:

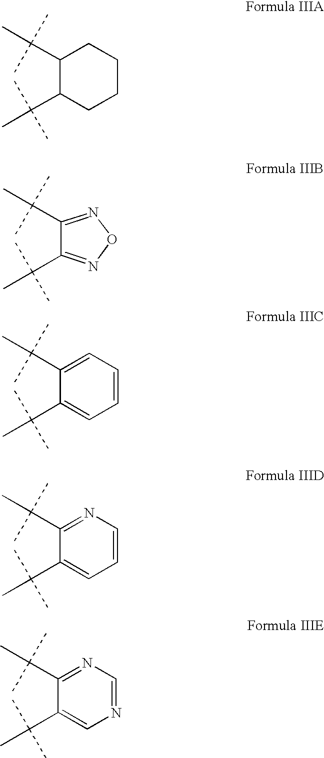

The inhibitors of Formula III comprising a ring A selected from IIIA, IIIB, IIIC, IIID, IIIE may further comprise:

R1 represents H, C1-C3 alkyl, aryl, alkylaryl, (CH₂)ₙCOR3, or (CH₂)ₙSO₂R3;
R3 represents H, C1-C4 alkyl, aryl, or alkylaryl;
R7 represents H, C1-C4 alkyl, halogens, NO₂, CF₃, aryl, carboxylate, aryloxy, amino, alkylamino, cyano, isocyanate, alkoxycarbonyl, or haloalkyl;
R8 represents H, C1-C4 alkyl, halogens, NO₂, CF₃, aryl, carboxylate, aryloxy, amino, alkylamino, cyano, isocyanate, alkoxycarbonyl, or haloalkyl; and
m=1, 2, 3.

In particular embodiments, alkylaryl is selected from Formula IIIF or IIIG:

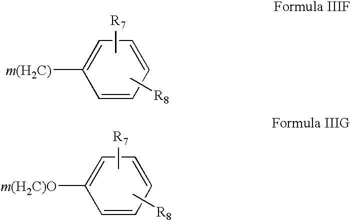

The inhibitors of Formula III may also be of the formula:

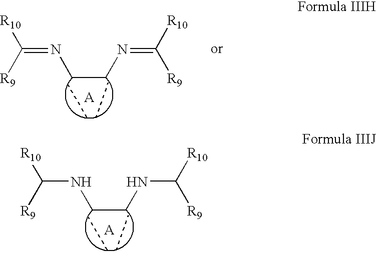

wherein,
A is a five or six member ring;
R9 represents H, C1-C3 alkyl, aryl, alkylaryl, (CH₂)ₙCOXR3, (CH₂)ₙXCOR3, (CH₂)ₙCOR3, CH₂(CH₂)ₙSO₂R3, CH₂(CH₂)ₙXR3, CH₂(CH₂)ₙSO₂XR3, or CH₂(CH₂)ₙXSO₂R3;
R10 represents H, C1-C3 alkyl, aryl, alkylaryl, (CH₂)ₙCOXR3, (CH₂)ₙXCOR3, (CH₂)ₙCOR3, CH₂(CH₂)ₙSO₂R3, CH₂(CH₂)ₙXR3, CH₂(CH₂)ₙSO₂XR3, or CH₂(CH₂)ₙXSO₂R3; and
R3, X, and n are as described for Formula III.

Ring A of inhibitors IIIH and IIIJ may be saturated, unsaturated or aromatic, and may optional be substituted with C and N.

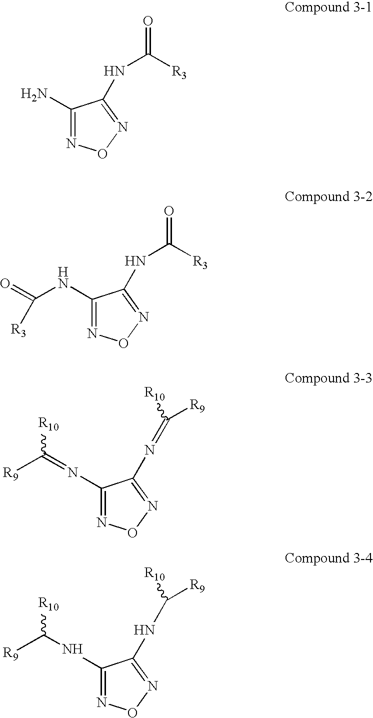

IV) Aryl Imidazole Carbonyl PTEN Inhibitors:

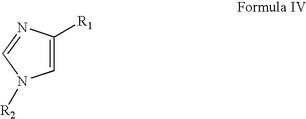

wherein,
R1 represents H, C1-C4 alkyl, aryl, alkylaryl, (CH₂)ₙCOXR3, (CH₂)ₘXCOR3, (CH₂)ₘXR3, (CH₂)ₙCOR3, (CH₂)ₙSO₂XR3, or (CH₂)ₘXSO₂R3;
R2 represents H, C1-C4 alkyl, aryl, or alkylaryl;
R3 represents H, C1-C3 alkyl, aryl, or alkylaryl;
R4 represents H, C1-C4 alkyl, aryl, alkylaryl, NHSO₂R5, NHCO₂R5, N═C(R5R6), or NR5R6;
R5 represents H, C1-C4 alkyl, aryl, or alkylaryl;
R6 represents H, C1-C4 alkyl, aryl, or alkylaryl;
m=1-3;
n=0-3; and
X represents O, NR4.

Compounds of formula IV may be of the formula:

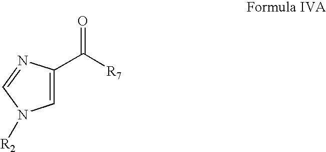

wherein, R7 represents XR4.

Compounds of formula IV may also be of the formula:

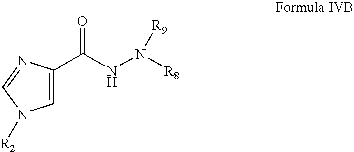

wherein
R8 represents H, C1-C4 alkyl, aryl, alkylaryl, (CH₂)ₙCOX—R3, (CH₂)ₙXCOR3, (CH₂)ₙX—R3, (CH₂)ₙCOR3, (CH₂)ₙSO₂XR3, or (CH₂)ₙXSO₂R3; and
R9 represents H, C1-C4 alkyl, aryl, alkylaryl.

Compounds of Formula IVB may also be:

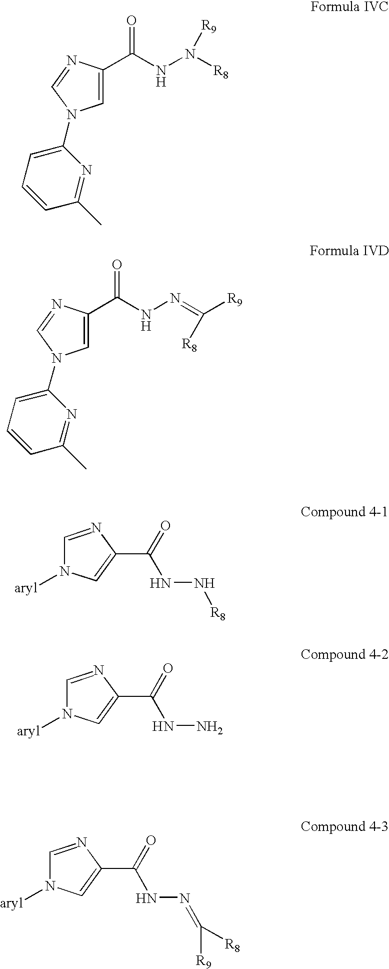

V) Polyamide PTEN Inhibitors

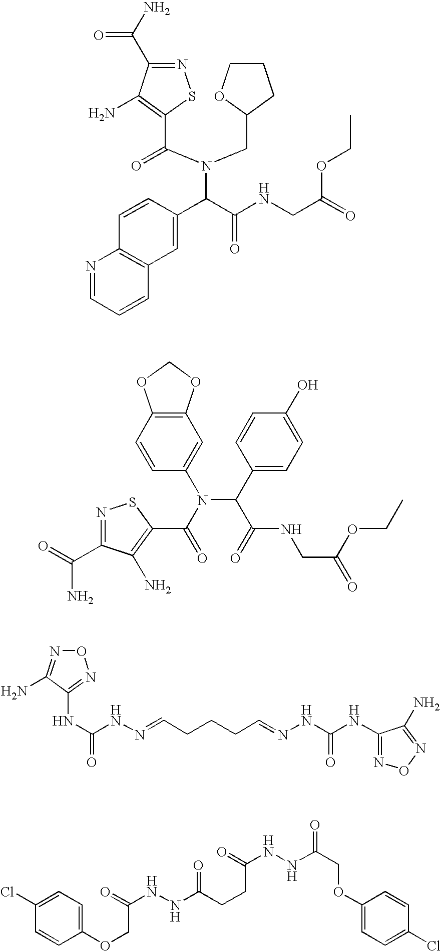

VI) Commercial PTEN Inhibitors
1. Deltamethrin; (S)-a-Cyano-3-phenoxybenzyl(1R)-cis-3-(2,2 dibromovinyl)-2,2-dimethylcyclopropanecarboxylate
2. Alendronate, Sodium, Trihydrate
3. N-(9,10-Dioxo-9,10-dihydrophenanthren-2-yl)-2,2-dimethylpropionamide
4. 5-Benzyl-3furylmethyl (1R,S)-cis,trans-chrysanthemate
5. Suramin, Sodium Salt; 8,8′-[carbonylbis[imino-3,1phenylenecarbonylimino (4-methyl-3,1-phenylene)carbonyliminol]]bis-, hexasodium salt
6. 4-Methoxyphenacyl Bromide
7. 1,4-Dimethylendothall; 1,4-Dimethyl-7-oxabicyclo[2.2.1]heptane-2,3-dicarboxylic Acid
8. Cantharidic Acid; 2,3-dimethyl-7-oxabicyclo[2.2.1]heptane-2,3dicarboxylic acid
9. Sodium Stibogluconate; Antimony Sodium Gluconate
10. 3,4-Dephostatin, Ethyl-
11. Fenvalerate; a-Cyano-3-phenoxybenzyl-a(4-chlorophenyl)isovalerate
12. α-Naphthyl Acid Phosphate, Monosodium Salt
13. β-Glycerophosphate, Disodium Salt, Pentahydrate
14. Endothall; 7-Oxabicyclo[2.2.1]heptane-2,3dicarboxylic Acid
15. Cypermethrin; (R,S)-α-Cyano-3-phenoxybenzyl-3(2,2-dichlorovinyl)-2,2-dimethylcyclopropanecarboxylate; (1R)-(R)cyano(3-phenoxyphenyl)methyl 3-(2,2-dichlorovinyl)-2,2-dimethylcyclopropanecarboxylate.

VII) 1,10-phenanthroline-5,6-dione PTEN Inhibitors

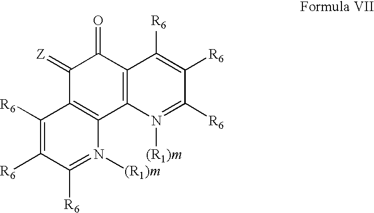

wherein,
R1 represents O, C1-C4 alkyl, (CH₂)ₙCOXR2, (CH₂)ₙXCOR2, (CH₂)ₙXR2, (CH₂)ₙCOR2, (CH₂)ₙSO₂XR2, (CH₂)ₙXSO₂R2, or (CH₂)ₙSO₂R2;
R2 represents H, C1-C4 alkyl, aryl, alkylaryl, NHSO₂R4, NHCOR4, NHCO₂R4, NHCOCO₂R4, or NR4R5;
R3 represents H, C1-C4 alkyl, aryl, alkylaryl, NHSO₂R4, NHCOR4, NHCO₂R4, NHCOCO₂R4, or NR4R5;
R4 represents H, C1-C4 alkyl, aryl, or alkylaryl;
R5 represents H, C1-C4 alkyl, aryl, or alkylaryl;
R6 at each occurrence is independently selected from hydrogen, halogen, NO₂, NR4R10, C1-C4 alkyl, NH(CH₂)ₚCO(CH₂)_{q}XR2, (CH₂)ₚCOXR2, (CH₂)ₚXCOR2, (CH₂)ₚXR2, (CH₂)ₚCOR2, (CH₂)ₚSO₂XR2, or (CH₂)ₚXSO₂R2;
R7 represents H, C1-C4 alkyl, aryl, alkylaryl, SO₂R4, NHSO₂R4, NHCO₂R4, or NR8R9;
R8 represents independently H, C1-C4 alkyl, aryl, alkylaryl, (CH₂)ₙCOXR2, or (CH₂)ₙXR2;
R9 represents independently H, C1-C4 alkyl, aryl, alkylaryl, (CH₂)ₙCOXR2, (CH₂)ₙXR2, (CH₂)ₚCOXR2, (CH₂)ₚXCOR2, (CH₂)ₚXR2, (CH₂)ₚCOR2, (CH₂)ₚSO₂XR2, (CH₂)ₚXSO₂R, or (CH₂)ₚSO₂R2;
R10 represents H, C1-C4 alkyl R7=H, C1-C4 alkyl, aryl, alkylaryl, SO₂R4, NHSO₂R4, NHCO₂R4, or NR8R9;
m represents independently 0 or 1;
n=1-5;
p=0-5;
q=0-5;
X represents O or NR3; and

### Z=O or NR7.

The nitrogen in the ring of compound of Formula VII may be neutral. The nitrogen may also be charged when bound to an R1 group (quaternary salt) in the case where at least one m=1.

The inhibitor may also be selected from:

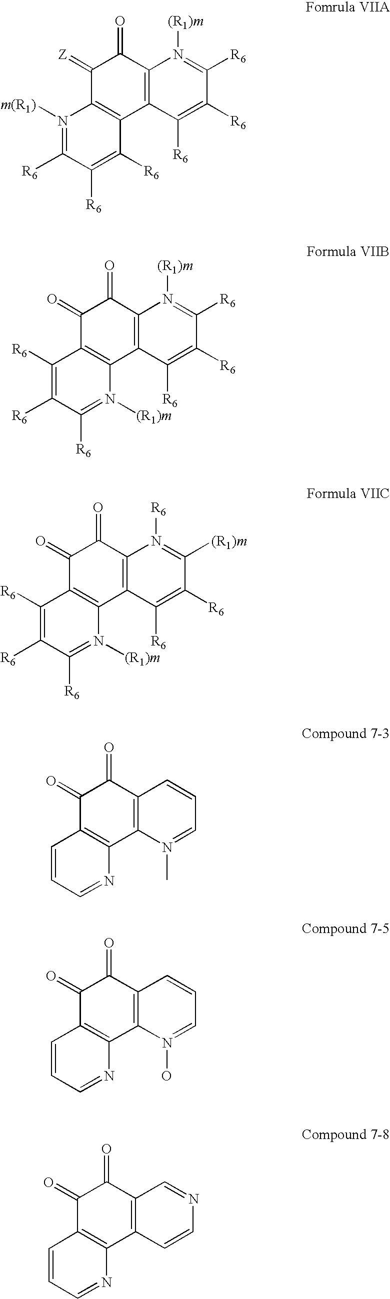

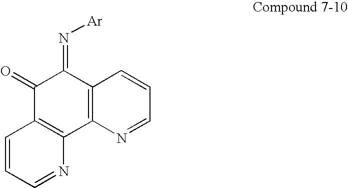

VIII) Substituted phenathrene-9-10-dione PTEN Inhibitors

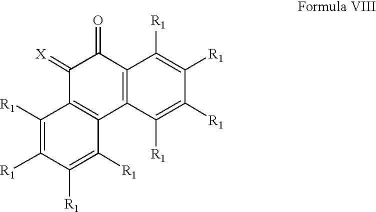

wherein,
R1 represents H, NO₂, NR5R6, halogen, cyano, alkyl, alkylaryl, carbonyl, carboxy, COR2, or CONR5R6;
R2 and R3 represent independently H, C1-C4 alkyl, aryl, or alkylaryl;
R4 represents H, C1-C4 alkyl, aryl, alkylaryl, SO₂—R2, NHSO₂R2, NHCOR2, NHCO₂R2, N═CR2R3, or NR5R6;
R5 represents H, C1-C4 alkyl, aryl, alkylaryl, (CH₂)ₙCOXR2, (CH₂)ₙXR2, (CH₂)ₙCO(CH₂)ₘXR2, SO₂R2, (CH₂)ₙCO(CH₂)ₙCOXR2, or (CH₂)ₙCOR2;
R6 represents H, C1-C4 alkyl, aryl, alkylaryl, (CH₂)ₙCOX—R2, (CH₂)ₙXR2, (CH₂)ₙCO(CH₂)ₘXR2, SO₂R2, (CH₂)ₙCO(CH₂)ₙCOXR2, or (CH₂)ₙCOR2;
m=0-3;
n=0-3; and
X represents CR2R3, O, NR4.

The inhibitors of Formula VIII may be of the formula:

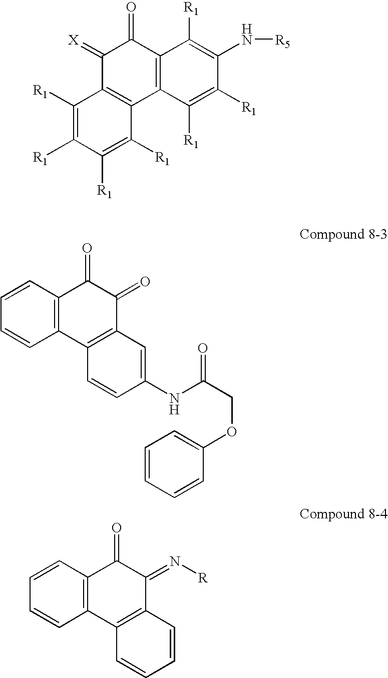

IX) Isatin (1H-indole-2,3-dione) PTEN Inhibitors

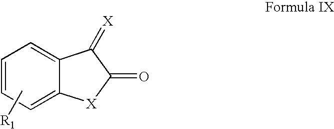

wherein,
R1 represents H, NO₂, NR5R6, halogen, cyano, alkyl, alkylaryl, carbonyl, carboxy, COR₂, CONR5R6, SO₃R2, or SO₂NR2R3;
R2 and R3 represent independently H, C1-C4 alkyl, aryl, or alkylaryl;
R4 represents H, C1-C4 alkyl, aryl, alkylaryl, SO₂—R2, NHSO₂R2, NHCOR2, NHCO₂R2, N═CR2R3, or NR5R6;
R5 represents H, C1-C4 alkyl, aryl, alkylaryl, (CH₂)ₙCOX—R2, (CH₂)ₙX—R2, (CH₂)ₙCO(CH₂)ₘXR2, SO₂R2, (CH₂)ₙCO(CH₂)ₙCOXR2, or (CH₂)ₙCOR2;
R6 represents H, C1-C4 alkyl, aryl, alkylaryl, (CH₂)ₙCOX—R2, (CH₂)ₙX—R2 (CH₂)ₙCO(CH₂)═XR2, SO₂R2, (CH₂)ₙCO(CH₂)ₙCOXR2, or (CH₂)ₙCOR2;
m=0-3;
n=0-3; and
X represents CR2R3, O, NR4.

The inhibitors of Formula IX may be selected from:

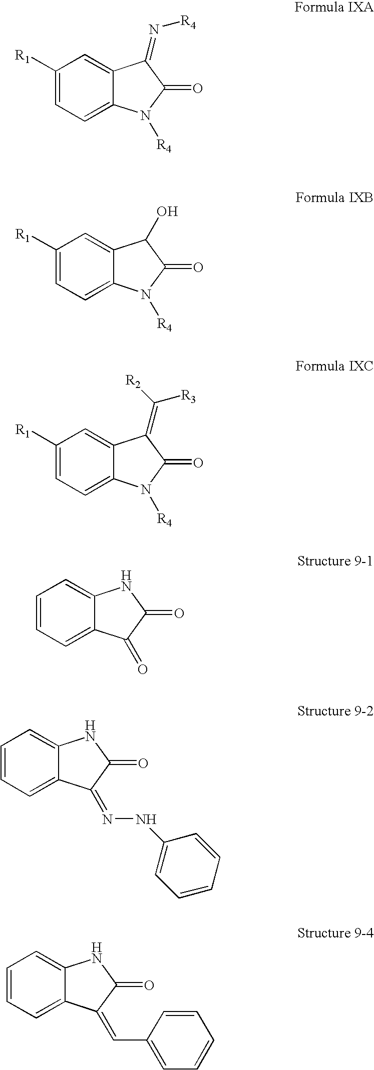

X) Substituted phenanthren-9-ol PTEN Inhibitors

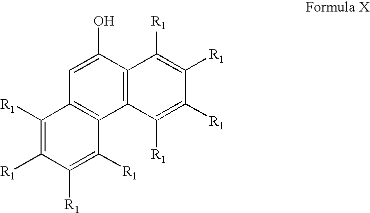

R1 represents H, N02, NR5R6, halogen, cyano, alkyl, alkylaryl, carbonyl, carboxy, COR2, or CONR5R6;
R2 and R3 represent independently H, C1-C4 alkyl, aryl, or alkylaryl;
R4 represents H, C1-C4 alkyl, aryl, alkylaryl, SO₂—R2, NHSO₂R2, NHCOR2, NHCO₂R2, N═CR2R3, or NR5R6;
R5 represents H, C1-C4 alkyl, aryl, alkylaryl, (CH₂)ₙCOXR2, (CH₂)ₙX—R2, (CH₂)ₙCO(CH₂)ₘXR2, SO₂R2, (CH₂)ₙCO(CH₂)ₙCOXR2, or (CH₂)ₙCOR2;
RO represents H, C1-C4 alkyl, aryl, alkylaryl, (CH₂)ₙCOX—R2, CCH₂)ₙX—R2, (CH₂)ₙCO(CH₂)ₘXR2, SO₂R2, (CH₂)ₙCO(CH₂)ₙCOXR2, (CH₂)ₙCOR2;
m=0-3;
n=0-3; and
X represents CR2R3, O, NR4.

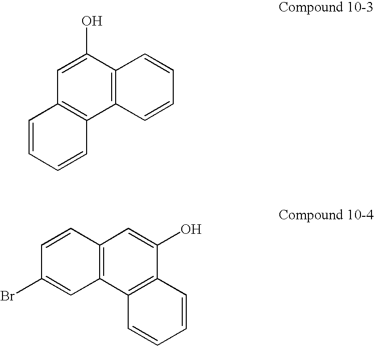

XI) Substituted naphthalene-1,2-dione PTEN Inhibitors

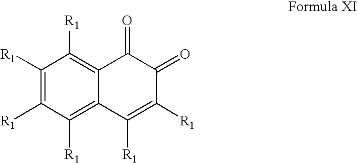

wherein,
R1 represents independently chosen from H, NO₂, NR3R4, halogen, cyano, alkyl, alkylaryl, carbonyl, carboxy, (CH₂)ₙCOXR3, COR2, SO₃—R2, SO₂N—R3R4, NHSO₂—R3, NHCO₂R3, NHCOR3, NHCOCO₂R2, NR3R4, or CONR3R4;
R2 represents H, C1-C4 alkyl, aryl, or alkylaryl;
R3 and R4 represent independently H, C1-C4 alkyl, aryl, alkylaryl, (CH₂)ₙCOXR2, (CH₂)ₙCO(CH₂)ₘXR2, or (CH₂)ₙOR2;
m=0-3;
n=0-3; and
X represents O, NR2.

The inhibitors of Formula XI may be selected from:

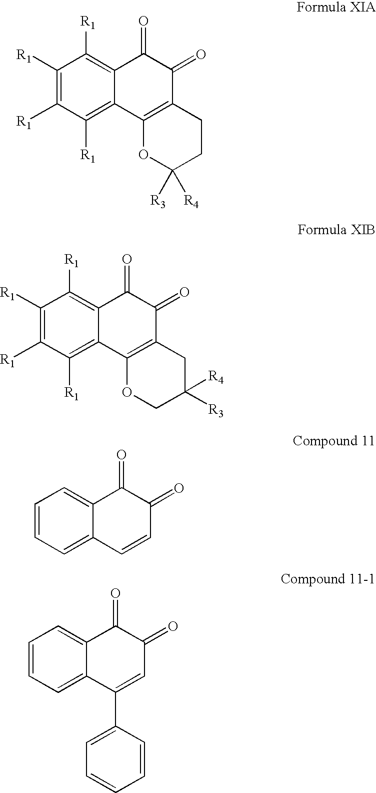

XII) Substituted naphthalene-1,4-dione PTEN Inhibitors

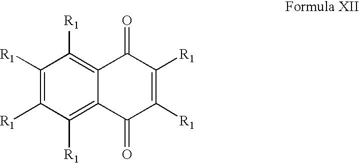

wherein
R1 represents H, NO₂, NR3R4, halogen, cyano, alkyl, alkylaryl, carbonyl, carboxy, (CH₂)ₙCOXR3, COR2, SO₃R2, SO₂N—R3R4, NHSO₂—R3, NHCO₂R3, NHCOR3, NHCOCO₂R2, NR3R4, or CON—R3R4;
R2 represents H, C1-C4 alkyl, aryl, or alkylaryl;
R3 and R4 represents independently H, C1-C4 alkyl, aryl, alkylaryl, (CH₂)ₙCOXR2, (CH₂)ₙCO(CH₂)ₘXR2, or (CH₂)ₙOR2;
m=0-3;
n=0-3; and
X represents O, NR2.

XIII) Vanadate-Based PTEN Inhibitors
1. Potassium Bisperoxo(bipyridine) oxovanadate (V)
2. Dipotassium Bisperoxo (5-hydroxypyridine-2-carboxyl) oxovanadate (V)
3. Dipotassium Bisperoxo (picolinato)oxovanadate (V)
4. Monoperoxo(picolinato) oxovanadate(V)
5. Potassiun Bisperoxo (1,10-phenanthroline) oxovanadate (V)
6. bis(N,N-Dimethylhydroxamido) hydroxooxovanadate

XIV) T1-Loop Binding Element Containing PTEN Inhibitors

The PTEN inhibitors may contain a group that exists at physiological pH in significantly anionic form, such as at least 5% of the molecular species at pH of 7.4 are anionic charged. Such anionic groups can bind to PTEN in the T1 loop of the peptide structure in solution.

Representative examples of such groups include:

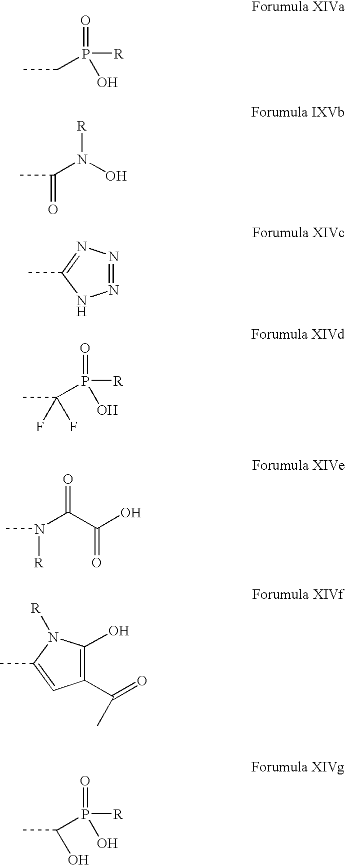

wherein R is independently chosen from H, OH, O-alkyl, alkyl, SH, S-alkyl, NH2, NH-alkyl, N-(alkyl)2 where alkyl is a small, C1-C4 alkyl moiety. The dashed lines represent the connection to the formulas of the compounds described for Formulas I through XIII above. The groups may be further evaluated in silico for their ability to fill the T1 loop space by standard molecular docking procedures. Such T1-loop binding groups may be incorporated into compounds of Formula I-XIII. Incorporation of the groups may impart selectivity of the molecules to inhibition of PTEN. Preparation of groups XIVa-XIVd are well established in the literature. Compounds of Formula XIVe may be prepared by methods disclosed in Wilson et al., Bioorganic & Medicinal Chemistry Letters, vol 6, No. 9, pp 1043-1046, 1996. Incorporation of these groups into the Formulas I-XIII is by standard synthetic methods easily attainable by those skilled in the art. Examples of such incorporation by simply utilizing appropriate starting materials is illustrated by the conversion of 7-9 to one incorporating the above groups, e.g.

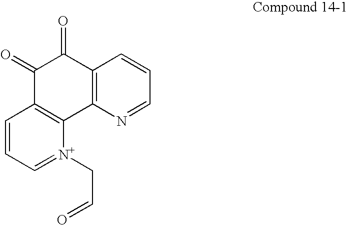

### Example 1

### Targeted Deletion of Tumor Suppressor PTEN Augments Neutrophil Function and Enhances Host Defense in Neutropenia-Associated Pneumonia: Accumulation of PTEN-Null Neutrophils in Inflamed Lungs is Enhanced in Bacterial Pneumonia

We recently reported that the responsiveness of neutrophils to chemoattractant stimulation is enhanced in PTEN-knockout mice in which PtdIns(3,4,5)P3 signaling is hyperactivated. Using a mouse peritonitis model, we showed that the recruitment of neutrophils to inflamed peritoneal cavity was highly elevated in these mice¹³. To investigate whether elevation of PtdIns(3,4,5)P3 signaling can also lead to increased neutrophil recruitment to inflamed lungs, we used a mouse bacterial pneumonia model. In this model, lung inflammation was induced by intratracheal instillation of the gram-negative bacterium E. coli, which is one of the most common pathogens in neutropenia-related pneumonia⁵⁻⁷. Because of the early embryonic lethality of conventional Pten ^{-/-} mice¹⁵, we used a conditional PTEN-knockout mouse, in which two loxP sequences were inserted on either side of exon 5 of the PTEN gene encoding the phosphatase domain. We crossed this mouse with a myeloid-specific Cre line, in which the Cre recombinase gene was under the control of a lysozyme M promoter. Thus, disruption of PTEN occurred only in mice with the myeloid linage, including monocytes, mature macrophages, and neutrophils¹². Mice that are homozygous for this allele are viable, fertile, normal in size, and do not display any gross physical or behavioral abnormalities¹³.

Neutrophil accumulation in inflamed lungs was assessed 8 and 24 hr after bacteria instillation by using two independent methods: bronchoalveolar lavage (BAL) and morphometric analyses of histological lung sections. Very few neutrophils were detected in the lungs of unchallenged mice. Similar to previously published data from other laboratories using the same model¹⁶⁻¹⁹, the number of neutrophils in the wild-type BAL fluid reached nearly 1×10⁶ cells/lung, 8 hr after bacteria instillation, and nearly 2×10⁶ cells/lung, 24 hr after bacteria instillation. Myeloid-specific PTEN^{-/-} mice showed a dramatic increase in bacteria-induced neutrophil recruitment. Nearly 2×10⁶ neutrophils were recruited to the lungs 8 hr after induction, and 3.5×10⁶ were recruited 24 hr after induction. We detected a similar effect when we quantified the number of emigrated neutrophils in alveolar air spaces by morphometric analyses of histological lung sections. Since PTEN disruption did not affect peripheral blood neutrophil count before or after bacteria instillation, the enhanced neutrophil accumulation is most likely due to elevated neutrophil recruitment and/or delayed neutrophil death in the inflamed lungs. In these experiments, specific increases in PtdIns(3,4,5)P3 signaling in recruited neutrophils was confirmed by measuring phosphorylation of Akt, a well known downstream factor in the PtdIns(3,4,5)P3 signaling pathway.

Dramatically increased lung neutrophil number often leads to aggravated lung damage. We consistently detected augmented pulmonary edema formation and increased protein accumulation in the lungs of the PTEN^{-/-} mice. In addition, a much increased pneumonia-associated death rate was observed in the PTEN^{-/-} mice. More than 80% of wild-type experimental mice survived the challenge. In contrast, only 50% of PTEN KO mice survived. Collectively, these results demonstrated that elevating PtdIns(3,4,5)P3 signaling by PTEN disruption can result in enhanced neutrophil accumulation and more severe lung inflammation in bacterial pneumonia in non-neutropenic mice. We also examined neutrophil accumulation and associated lung inflammation in pneumonia induced by sterile ligands LPS. Essentially the same results were observed—PTEN disruption led to elevated neutrophil recruitment and aggravated lung inflammation.

### Example 2

### Augmenting the PtdIns(3,4,5)P3 Signal by Disrupting PTEN Delays Neutrophil Death in Bacterial Pneumonia

One mechanism that can lead to enhanced pulmonary neutrophil accumulation is increasing the lifespan of recruited neutrophils. Neutrophils are terminally differentiated and usually have a short lifespan (1-4 days in tissue). They die via programmed cell death (apoptosis). We have shown that augmenting the PtdIns(3,4,5)P3 signal by depleting PTEN dramatically delays the spontaneous death of cultured neutrophils¹³. In this study, we explored whether neutrophil death in inflamed lungs is also delayed in PTEN-knockout mice. We first measured the apoptosis of neutrophils collected from bronchoalveolar lavage fluids (BALF). Few apoptotic neutrophils were detected in wild-type or PTEN-null mice. In the inflamed lung, the apoptotic neutrophils might have already been cleared by macrophages before they could emigrate into the alveolar space. Thus, we checked the apoptosis of neutrophils in the lung tissues. The viability of neutrophils in histological lung sections was determined by a terminal deoxynucleotide transferase dUTP nick end-labeling (TUNEL) assay, which detects fragmented DNA in apoptotic cells^{20,21}. In wild-type mice, about 10% of recruited pulmonary neutrophils were TUNEL positive 24 hr after the bacteria instillation. This number dropped to 7% in PTEN-knockout mice, indicating reduced neutrophil death in these mice.

To further investigate whether the reduction of in vivo neutrophil death was due to alterations of the intrinsic apoptotic/survival pathway in the PTEN-null neutrophils or due to an altered lung inflammatory environment in the myeloid-specific PTEN-knockout mice, we conducted an adoptive transfer experiment using a mouse peritonitis model. We labeled purified PTEN-null neutrophils with intracellular fluorescent dye [5-(and -6)-carboxyfluorescein diacetate succinimidyl esters (CFSE) (green)] and wild-type neutrophil with another dye [5-(and -6)-chloromethyl SNARF-1 acetate (red)] or vice versa. The mixed (1:1) population was injected into the peritoneal cavity of the same wild-type mice. The relative rate of apoptosis was calculated by measuring the ratio between PTEN-null and wild-type neutrophils at the inflammation site. By doing this, we skipped the step of neutrophil recruitment. Thus the number of neutrophils in the peritoneal cavity will directly reflect the death rate of each population. In addition, because the wild-type and PTEN-null neutrophils were in exactly the same environment, variability caused by the difference of each individual recipient mouse was eliminated. PTEN-null and wild-type neutrophils were identified by their unique fluorescent labels using FACS analysis. Supporting our conclusions that PTEN-null neutrophils have a prolonged lifespan, we detected greatly delayed clearance of transplanted PTEN-null neutrophils compared with wild-type neutrophils.

### Example 3

### Augmenting PtdIns(3,4,5)P3 Signal by Depleting PTEN Enhances the Bacteria-Killing Capability of Neutrophils

In the mouse bacterial pneumonia model, we detected elevated neutrophil recruitment to inflamed lungs in PTEN-null mice. We subsequently explored the survival rate of intratracheally instilled live E. coli cells. Due to cell proliferation, the number of bacteria gradually increased after initial instillation. When a significant number of neutrophils accumulated in the lungs (8-24 hrs after the instillation), the number of bacteria stopped increasing, reflecting the bacteria-killing capability of neutrophils. We detected fewer bacteria in inflamed myeloid-specific PTEN-null mice, suggesting that these mice have enhanced bacteria-killing capability. This finding could be the result of elevated neutrophil recruitment detected in PTEN-null mice. To test whether the intrinsic bacteria-killing capability of neutrophils is also enhanced by PTEN disruption, we conducted an in vitro assay using the same number of wild-type and PTEN-null neutrophils. The bacteria-killing capability of PTEN-null neutrophils was increased by 40% at 30 min and 60% at 2 hr, compared with wild-type neutrophils. Augmented phagocytosis could be responsible for enhanced bacteria killing. To test this, we quantified the number of bio-particles engulfed by each neutrophil using an in vitro phagocytosis assay. After 1 hr incubation at 37° C., an average of 60 mouse serum-opsonized fluorescein-conjugated zymosan particles were engulfed by 100 wild-type neutrophils (phagocytic index). PTEN^{-/-} neutrophils had a dramatically increased phagocytic index: nearly 100 bacteria were engulfed by 100 neutrophils. A similar effect was detected when the in vitro phagocytosis assay was conducted using purified mouse neutrophils and serum-opsonized fluorescein-conjugated bacteria bioparticles. These results are consistent with a previous report indicating an increased phagocytosis in PTEN-null macrophages²². The augmented phagocytosis was likely a result of enhanced engulfment, since there was essentially no difference in the initial bacteria/zymosan-binding capability between wild-type and PTEN-null neutrophils. We also monitored phagosome-lysosome fusion using a Lysotracker fluorescent dye. No obvious alteration was detected in the PTEN null neutrophils, indicating that elevation of PtdIns(3,4,5)P3 signal does not affect phagosome maturation.

Elevated bacteria killing capability could also be a result of enhanced superoxide production in phagosomes. Accordingly, we measured phagocytosis-associated superoxide production in both PTEN knockout and WT neutrophils. A significant enhancement was observed in the PTEN null neutrophils for both E. coli and Zymosan-induced superoxide production. Recently, Karen et al reported that superoxide production elicited by serum-opsonized Bioparticles is mainly mediated by complement and CD18. Interestingly, this process requires class III PI3K and its product PtdIns(3)P, and is independent of class I PI3K and its product PtdIns(3,4,5)P3²³. PTEN acts as a lipid phosphatase, removing the phosphate in the D3 position of the inositol ring from PtdIns(3,4,5)P3, PtdIns(3,4)P2, and PtdIns(3)P. Thus its effect on phagocytosis-associated superoxide production is most likely mediated by its lipid phosphatase activity on PtdIns(3)P. Finally, we measured neutrophil intracellular bactericidal activity using a gentimicin protection assay. Consistent with the elevated superoxide production, the capability of the PTEN-null neutrophils to kill engulfed bacteria was much increased compared to the WT neutrophils. Together, these findings further demonstrate the enhanced bacteria-killing capability of neutrophils in which the PtdIns(3,4,5)P3 signal is augmented.

### Example 4

### Disruption of PTEN Enhances Pulmonary Neutrophil Accumulation and Reduces Bacterial Burden in Neutropenia-Associated Pneumonia

Excessive neutrophil accumulation or hyper-responsiveness of neutrophils can damage surrounding tissues and cause unwanted and exaggerated tissue inflammation. However, neutrophils are the major cell type in innate immunity, and they protect their host by engulfing, killing, and digesting invading bacterial and fungal pathogens. Thus, elevated neutrophil function might be beneficial in certain pathological conditions. Accordingly, we investigated whether enhancing neutrophil function by PTEN disruption can be used as a therapeutic strategy to augment host defense in neutropenia-related pneumonia. We first examined neutrophil recruitment and its bacteria-killing capability under neutropenic conditions. We induced neutropenia using a widely used chemotherapeutic drug, cyclophosphamide (Cy), which is used primarily to treat cancer. The mechanism of action is thought to involve cross-linking and strand breakage of tumor cell DNA^{24,25}. The induction of neutropenia by Cy has been well documented²⁶⁻²⁸. Two intraperitoneal injections for a total dose of 250 mg/kg (150 mg/kg at day 1 and 100 mg/kg at day 4) were sufficient to induce a severe neutropenia. On day 5, Cy-treated mice had approximately 90% fewer circulating neutrophils than the untreated group. The profound neutropenia persisted through days 6 and 7. Due to the severe neutropenia, some wild-type and PTEN-knockout mice can not survive bacterial challenge at a dose of 10⁶ cfu/mouse. Thus, we used 10⁵ cfu/mouse in all experiments involving neutropenic mice. After bacterial instillation, the number of neutrophils accumulated in the lungs was significantly lower in both wild-type and myeloid-specific PTEN-null neutropenic mice, compared with untreated mice. However, PTEN-null mice still had about 3-fold more bacteria-induced pulmonary neutrophil accumulation compared with wild-type mice. Consistently, the activity of myeloperoxidase (MPO), a peroxidase enzyme most abundantly present in phagocytes, was substantially elevated in the lungs of PTEN-null mice. Moreover, the enhanced neutrophil accumulation in the PTEN-null mice led to better clearance of instilled bacteria, as revealed by the reduced bacteria number in the inflamed lungs of these mice. As a result, the resolution of bacteria-induced lung inflammation was accelerated. The inflammation-associated lung damage, which was evaluated by pulmonary edema formation, was alleviated in PTEN-null mice. We also examined neutrophil recruitment, bacteria-killing capability, and lung inflammation in irradiation-induced neutropenic mice; and essentially the same results were obtained, demonstrating that the protective effects detected in the PTEN-null mice were not “model-dependent”.

### Example 5

### Disruption of PTEN Directly Increases the Efficiency of Neutrophil Recruitment to the Inflamed Lungs

We have shown that the accumulation of neutrophils in both inflamed lungs and inflamed peritoneal cavity¹³ was enhanced in myeloid-specific PTEN-null mice. We further demonstrated that prolonged neutrophil survival is at least partially responsible for this elevated accumulation. However, whether the efficiency of neutrophil recruitment to the sites of inflammation is also increased in the myeloid-specific PTEN-null mice has not been directly examined in vivo. Thus, we next investigated neutrophil recruitment in neutropenic mice by using an adoptive transfer assay. We labeled in vitro purified PTEN-null neutrophils with CFSE and wild-type neutrophils with SNARF-1 acetate, or vice versa. The mixed (1:1) population was intravenously injected into a wild-type neutropenic recipient mouse 2.5 hr after the intratracheal instillation of E. coli. By doing this, we were able to compare the recruitment of these two types of neutrophils in exactly the same environment. Independent of the dye used to stain the neutrophils, we consistently detected enhanced (more than two-fold) pulmonary recruitment of PTEN-null neutrophils compared with wild-type neutrophils. This result indicates that the observed elevation of neutrophil accumulation in PTEN-null mice is a combination of enhanced neutrophil recruitment and delayed neutrophil death.

### Example 6

### Disruption of PTEN Increases the Recruitment of Macrophages in the Inflamed Lungs

Besides neutrophils, alveolar macrophages are also considered major effector cells in host defense against respiratory tract infections by virtue of their potent phagocytic properties²⁹⁻³³. They are also important in initiating the inflammatory responses. In response to danger, alveolar macrophages produce various proinflammatory mediators to orchestrate the inflammatory response, leading to the recruitment of other types of cells, including neutrophils, to the lungs^{34,35}. In the myeloid specific PTEN knockout mice used in this study, PTEN expression is also ablated in macrophages¹³. Thus, it is possible that elevation of PtdIns(3,4,5)P3 signaling can also alter the recruitment and function of macrophages in neutropenia-related pneumonia. To test this, we first measured the number of alveolar macrophages in resting and inflamed lungs. Two populations of macrophages have been identified in the mouse, resident macrophages and inflammatory macrophages³⁶⁻⁴⁰. The extravasation of resident macrophages can occur in un-inflamed lungs and is independent of neutrophils, while the recruitment of inflammatory macrophages to inflamed lungs occurs after infection and is a neutrophil dependent process⁴¹. Since both macrophage populations play crucial role in host defense and bacterial killing, we measured the number of alveolar macrophages both before and after bacteria challenge.

The number of resident alveolar macrophages in BAL fluid of unchallenged mice were accessed by Wright-Giemsa staining in which alveolar macrophages were identified by their large size, large cytoplasmic region and single, round nucleus, and FACS analysis in which resident alveolar macrophages were identified as CD11b⁻F4/80⁺ cells⁴². This number was increased by more than 60% in the PTEN knockout mice in both assays. During lung inflammation, recruitment of inflammatory macrophages occurs after neutrophil recruitment, and usually peaks at 24-48 hr after bacterial infection. Accordingly, we measured the amount of inflammatory macrophages in inflamed lungs at 24 hr. We used FACS analysis to more precisely characterize the population of macrophages. Briefly, BAL fluid cells were gated according to their FSC versus SSC characteristics thereby excluding contaminating red blood cells and cell debris. This was followed by hierarchical sub-gating according to their CD11b versus F4/80 antigen expression. Resident alveolar macrophages were then identified as F4/80⁺, and CD11b⁻ cells. Inflammatory induced exudate macrophages were identified as F4/80⁺, and CD11b^{hi} cells. As previously reported⁴², the total number of alveolar macrophages decreased after bacterial challenge. However, similar with neutrophils recruitment during lung inflammation, the recruitment of inflammatory macrophages was dramatically elevated in the PTEN knockout mice.

### Example 7

### Cytokine and Chemokine Production was Increased in the PTEN Knockout Mice

Inflammation is always associated with a large amount of cytokine and chemokine release from the site of infection. These cytokines and chemokines can subsequently induce accumulation and activation of neutrophils, monocytes/macrophages, eosinophils and lymphocytes. They also directly facilitate the clearance of pathogens by immune cells. In PTEN knockout mice, the enhanced recruitment of neutrophils to the inflamed lungs could also be a result of elevated pro-inflammatory cytokine/chemokine levels in the lungs. Neutrophil recruitment to the inflamed lungs is mainly mediated by CXCR2 receptor⁴³⁻⁴⁶. Keratinocyte-derived cytokine (KC) and macrophage-inflammatory protein-2 (MIP-2) are two of the CXCR2 receptor ligands in mouse and their levels in the lungs increase dramatically during the course of pneumonia⁴⁷⁻⁵⁰. PTEN disruption may directly increase the production of these chemokines in the inflamed lungs. Alternatively, PTEN disruption may enhance neutrophil recruitment via affecting some other early cytokines, such as TNF-a, IL-6, and IL-1, which are rapidly induced by bacteria and promote neutrophil recruitment indirectly. It was reported that neutrophil recruitment was significantly decreased by combined deficiency of TNF-a and IL-1 signaling⁵¹⁻⁵⁴. Accordingly, we measured the level of these chemokine/cytokines in the inflamed lungs of both WT and PTEN KO mice. In BAL fluids collected at 24 hr after E. coli instillation, the concentrations of all 5 cytokines/chemokines were significantly increased in the PTEN KO mice, demonstrating that the elevated neutrophil recruitment in these mice is also partially contributed by the elevated cytokine/chemokine levels.

Among the known CXCR2 ligands, macrophages are thought to be important sources for MIP-2 and KC⁴⁷⁻⁵⁰. Activated macrophages are also a major source of some early cytokines such as TNFa, and IL-6 in inflamed lungs. Thus, we directly examined whether PTEN disruption in macrophages can affect the LPS-induced production of MIP-2, KC, TNFa and IL-6 using an in vitro assay. Resident alveolar macrophages were prepared from WT and PTEN knockout mice and stimulated with LPS for 24 hours. Our results showed that PTEN disruption didn't increase the production of cytokine/chemokines by macrophages. Interestingly, PTEN depletion even led to a very small but statistically significant decrease of TNFa and IL-6 production. Collectively, these results indicate that the elevated cytokine/chemokine levels in the BALF are mainly caused by the increased number of resident alveolar macrophages, and not by their enhanced capability of producing cytokines or chemokines.

### Example 8

### Disruption of PTEN Alleviates the Severity of and Decreases the Mortality Associated with Neutropenia-Related Pneumonia

We demonstrated that augmentation of the PtdIns(3,4,5)P3 signal by depleting PTEN enhances neutrophil/macrophage recruitment to the inflamed lungs and increases the bacteria-killing capability of the recruited neutrophils in both normal and neutropenic mice. As a result, the bacterial burden in the infected myeloid-specific PTEN-knockout mice was reduced. Since the direct cause of neutropenia-related pneumonia is the lack of neutrophils in the infected lungs to clear the invading bacteria, the enhanced phagocyte accumulation and the augmented bacteria-killing capability in PTEN-knockout mice should lead to a quicker resolution of lung inflammation under neutropenic conditions. This was supported by the reduced formation of pulmonary edema in bacteria-challenged neutropenic PTEN-null mice. To provide more direct evidence, we conducted an X-ray radiographic analysis to assess the severity of lung inflammation. In wild-type mice, bacterial instillation induced severe inflammation in 24 hr, as shown by pulmonary infiltrates and abnormal diffuse radiographic opacities throughout the lungs. In contrast, the inflammation in the PTEN-null mice was less severe and usually lasted for less than 24 hr. Severe pneumonia is often accompanied with vascular leakage. BAL protein level increase has been used as an indicator of vascular leakage and a key parameter of inflammatory lung injury. Consistent with the enhanced bacteria killing capability and the alleviated lung inflammation in the PTEN knockout mice, we detected much decreased total protein level in BAL fluid in the inflamed lungs. Pneumonia is also accompanied by compromised lung mechanics^{52,55}; thus, we further explored the integrity of lung function by measuring pulmonary compliance and resistance using an invasive monitoring method. Lung compliance, which is measured as the pulmonary volume change per unit of pressure change, reflects the comparative stiffness or elasticity of the lung—the stiffer the lung, the lower the compliance. Compliance is often reduced by edema in the alveolar spaces during lung inflammation. Lung resistance, which is the amount of pressure required to cause a unit change of gas flow, reflects both narrowing of the conducting airways and parenchymal viscosity. In the absence of pneumonia, there were no significant differences in pulmonary compliance and resistance between wild-type and myeloid-specific PTEN-null mice. As expected, lung compliance decreased and pulmonary resistance increased in mice with pneumonia. However, such alterations in lung mechanics were not as pronounced in neutropenic PTEN-null mice compared with their wild-type littermates at 24 hour after E. coli instillation, indicating that disruption of PTEN alleviates the severity of lung inflammation in neutropenic mice.

Lastly, severe neutropenia-related pneumonia can lead to death. Thus, we investigated whether augmentation of the PtdIns(3,4,5)P3 signal by depleting PTEN can reduce such lethality. Consistent with the X-ray and lung function data, the increased bacteria-clearance capability and less severe inflammation in the myeloid-specific PTEN-null mice resulted in a higher survival rate of bacteria-challenged neutropenic mice. More than 50% of PTEN experimental mice survived the challenge. In contrast, only 7% of wild-type mice survived. In above experiments, the mouse neutropenia was induced with chemotherapeutic drug Cy. We also examined pneumonia-associated lethality of irradiation-induced neutropenic mice. Essentially the same results were obtained—PTEN disruption led to a higher survival rate of neutropenic mice, demonstrating that the protective effect observed in the PTEN-null mice was not a “model-dependent” phenomenon. Taken together, these observations provide direct evidence that augmenting PtdIns(3,4,5)P3 by disrupting PTEN can alleviate the severity of neutropenia-related pneumonia. This effect is mainly mediated by the elevated bacterial killing capability, which is largely due to the augmented recruitment and enhanced function of neutrophils. Supporting this idea, when the neutropenia-related pneumonia was induced by sterile ligands LPS, we could not detect statistically significant difference in the survival rate between the WT and PTEN KO neutropenic mice. The elevated neutrophil recruitment was still observed in the PTEN KO mice. Nevertheless, this led to even more severe lung damage, instead of alleviation of lung inflammation as observed in the pneumonia induced by live E. coli.

### Example 9

### Cyclophosphamide and Irradiation-Induced Mouse Neutropenia Model

Cyclophosphamide powder (Cytoxan®, Bristol-Myers Squibb, Princeton, N.J.) was dissolved in distilled water for injection at a final concentration of 20 mg/ml. Cyclophosphamide was injected intraperitoneally at a total dose of 250 mg/kg (two 0.5-mL injections on day 1 (150 mg/kg) and day 4 (100 mg/kg). Blood samples (˜30 μL) were taken from the retro-orbital sinuses of anesthetized uninfected mice by using heparinized capillary tubes (Modulohm A/S, Herlev, Denmark) on days 1, 4, 5, 6, and 7. Total and differential white blood cell counts (neutrophils, lymphocytes, and monocytes) were performed using a Hemavet 850 hematology system (Drew-Scientific Inc, Ramsey, Minn.), which is a multiparameter, automated hematology analyzer designed for in vitro diagnostic use. For irradiation-induced neutropenia, mice were exposed to a single dose of 600 cGy in a Gammacell 40 ¹³⁷Cs Irradiator (MDS Nordion, Ottawa, Canada). Total and differential white blood cell counts were monitored on days 3, 4, 5, 6.

### Example 10

### Pharmacological Inhibition of PTEN Enhances Pulmonary Neutrophil Accumulation, Reduces Bacterial Burden and Alleviates the Severity of and Decreases the Mortality Associated with Neutropenia-Associated Pneumonia

Here we show that enhancing neutrophil function by PTEN pharmacological inhibition can be used as a therapeutic strategy to augment host defense in neutropenia-related pneumonia. In preliminary experiments we demonstrated pharmacological inhibition of PTEN in neutrophils using a panel of small-molecule PTEN inhibitors. We also examined neutrophil recruitment and its bacteria-killing capability under neutropenic conditions. We induced neutropenia using a widely used chemotherapeutic drug, cyclophosphamide (Cy). Two intraperitoneal injections for a total dose of 250 mg/kg (150 mg/kg at day 1 and 100 mg/kg at day 4) were sufficient to induce a severe neutropenia. On day 5, Cy-treated mice had approximately 90% fewer circulating neutrophils than the untreated group. The profound neutropenia persisted through days 6 and 7. As noted above, due to the severe neutropenia, some mice can not survive bacterial challenge at a dose of 10⁶ cfu/mouse, so we used 10⁵ cfu/mouse in all experiments involving neutropenic mice.

PTEN inhibitors prepared in stock solutions of 25 mM in DMSO are serially diluted (5× each) in PBS to achieve the indicated 0.2 uM, 1.0 uM 5.0 uM, and 25.0 uM final concentrations (50 μl in 1-2 ml blood volume):

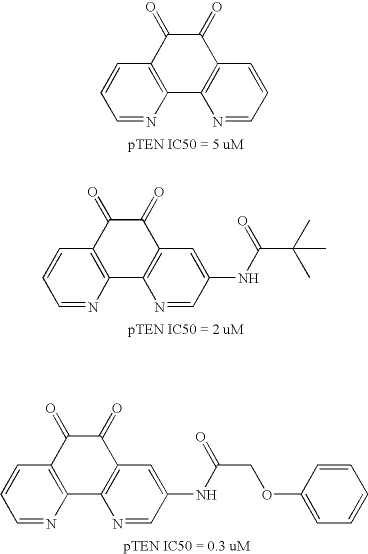

PTEN inhibitors SF1670 and D2 were obtained from Sheng Ding, The Scripps Research Institute, 2009. The PTEN inhibitors are administered intravenously (50 μl injected intravenously with a 28-gauge, 0.5-in. needle into the tail vein of each mouse) on day −1 from bacterial instillation (pre-treatment group) or day +1 from bacterial instillation (post-treatment group).

Bacterial colonies in histological lung sections are quantified and expressed as colony numbers in each 400× field. Bacterial killing in inflamed lungs: live bacteria in lung homogenates are assessed with a colony assay, wherein purified bone marrow neutrophils are incubated with E. coli for 2 hr. Diluted aliquots are spread on agar plates and incubated overnight at 37° C. In vitro bacterial killing capabilities are reflected by the decrease of colony-forming units after indicated incubation periods. Pulmonary edema formation is quantified as the percentage of edema in the total parenchymal region using IPlab Imaging software.

Both pre- and post-treatment groups have 2-5-fold more bacteria-induced pulmonary neutrophil accumulation compared with our control group. Consistently, the activity of myeloperoxidase (MPO), a peroxidase enzyme most abundantly present in phagocytes, is substantially elevated in the lungs of both treatment groups. Moreover, the enhanced neutrophil accumulation in the treated mice led to better clearance of instilled bacteria, as revealed by reduced bacteria number in the inflamed lungs of these mice. As a result, the resolution of bacteria-induced lung inflammation is accelerated. Inflammation-associated lung damage, evaluated by pulmonary edema formation, is alleviated in both groups of PTEN-inhibitor treated mice. Neutrophil recruitment, bacteria-killing capability, and lung inflammation in irradiation-induced neutropenic mice yield essentially the same results, demonstrating that the protective effects detected in the PTEN-inhibitor treated mice are not “model-dependent”.

### Example 11

### Exemplary PTEN Inhibition Assays for General Screening and IC50 Determinations

PTEN inhibitors are evaluated in an inhibition assay conducted in half-volume 96 well plates in 25 ul total volume per well containing 2 mM dithiothreitol (DTT) and 0.1 mM Tris buffer, pH 8.0 and up to 3 ug total protein of PTEN. Small volumes of the test inhibitor candidates (stock concentrated solutions of 25 mM in DMSO) are mixed with the PTEN solution at room temperature for about 10 minutes and then substrate is added. The reaction mix is then incubated in 37° C. for 20 minutes. Subsequent to this a 100 ul aliquot of malachite green buffer (Upstate, Charlottesville, Va.) is added to develop the color in the dark at room temperature (this solution also stops the dephosphorylation reaction). A SpectraMax Plus spectrophotometric plate reader (Molecular Devices, Sunnyvale, Calif.) is used to measure the optical density at 650 nanometers.

The initial screening concentration of inhibitor candidates is 250 uM and candidates with inhibition greater than 50% compared with a no-inhibitor control group are then evaluated further to determine IC50 values. PTEN can be purchased commercially or prepared by literature methods [i.e. from cell extracts of bacteria expressing genetic reconstituted Glutathione-5-transferase (GST)-PTEN fusion protein whereupon the GST-PTEN in the cell extract is bound onto and purified from Glutathione Sepharose 4B gel (Amersham, Piscataway, N.J.)]. Suitable PTEN reaction substrates include (a) PIP3 Phospholipid vesicle (PLV), which may be made using published methods (Maehama et al. 2000, Analytical Biochemistry 279, 248-250) and is typically utilized at about 50 uM in the final reaction mixture (based on component concentration), (b) water soluble PIP3 Echelon Biosciences, Salt Lake City, Utah, utilized at a working concentration of 100 uM, and (c) phosphorylated poly glutamic-tyrosine peptide designated (EEEEYp)n, where n=2 or 3 (Biofacilities of Indiana University, Indianapolis, Ind.), wherein a working concentration of the phosphorylated tyrosine substrate is 200 uM.

To determine the dose response of potential PTEN inhibitors, doses of test compounds ranging from 1 nM to 250 uM (final reaction mix concentrations) are evaluated in the general PTEN inhibition assay (supra). To obtain performed IC50 data, two separate rounds of the dose response assay are performed. In the first round, PTEN activity is tested in the presence of inhibitor at 10 fold serial dilutions ranging from 1 nM to 250 uM. Once the concentration range is determined, at which PTEN activity changes dramatically, two additional concentration data points within this range are added and the PTEN inhibition assay is then rerun for the second round. The PTEN inhibition IC50 is presented as the inhibitor concentration at which 50% of the PTEN activity (measured by phosphate production and compared to un-inhibited control samples) is found. The IC50 determination from the data is made using Prism software (GraphPad Software, San Diego, Calif.).

### REFERENCES

1. Hoare Z, Lim W S. Pneumonia: update on diagnosis and management. Bmj. 2006; 332:1077-1079.
2. Loeb M. Community acquired pneumonia. Clin Evid. 2006:2015-2024.
3. Ostendorf U, Ewig S, Torres A. Nosocomial pneumonia. Curr Opin Infect Dis. 2006; 19:327-338.
4. Mizgerd J P. Acute lower respiratory tract infection. N Engl J. Med. 2008; 358:716-727.
5. Joos L, Tamm M. Breakdown of pulmonary host defense in the immunocompromised host: cancer chemotherapy. Proc Am Thorac Soc. 2005; 2:445-448.
6. Viscoli C, Varnier O, Machetti M. Infections in patients with febrile neutropenia: epidemiology, microbiology, and risk stratification. Clin Infect Dis. 2005; 40 Suppl 4:S240-245.
7. Leung A N, Gosselin M V, Napper C H, et al. Pulmonary infections after bone marrow transplantation: clinical and radiographic findings. Radiology. 1999; 210:699-710.
8. lijima M, Huang Y E, Devreotes P. Temporal and spatial regulation of chemotaxis. Dev Cell. 2002; 3:469-478.
9. Stephens L, Ellson C, Hawkins P. Roles of PI3Ks in leukocyte chemotaxis and phagocytosis. Curr Opin Cell Biol. 2002; 14:203-213.
10. Ridley A J, Schwartz M A, Burridge K, et al. Cell migration: integrating signals from front to back. Science. 2003; 302:1704-1709.
11. Boume H R, Weiner O. A chemical compass. Nature. 2002; 419:21.
12. Zhu D, Hattori H, Jo H, et al. Deactivation of phosphatidylinositol 3,4,5-trisphosphate/Akt signaling mediates neutrophil spontaneous death. Proc Natl Acad Sci USA. 2006; 103:14836-14841.
13. Subramanian K K, Jia Y, Zhu D, et al. Tumor suppressor PTEN is a physiologic suppressor of chemoattractant-mediated neutrophil functions. Blood. 2007; 109:4028-4037.
14. Jia Y, Subrahmanyam K K, Emeux C, et al. Inositol 1,3,4,5-tetrakisphosphate negatively regulates PtdIns(3,4,5)P3 signaling in neutrophils. Immunity. 2007; 27:453-467.
15. Groszer M, Erickson R, Scripture-Adams D D, et al. Negative regulation of neural stem/progenitor cell proliferation by the Pten tumor suppressor gene in vivo. Science. 2001; 294:2186-2189.
16. D'Hulst A I, Vermaelen K Y, Brusselle G G, Joos G F, Pauwels R A. Time course of cigarette smoke-induced pulmonary inflammation in mice. Eur Respir J. 2005; 26:204-213.
17. Dallaire F, Ouellet N, Simard M, Bergeron Y, Bergeron M G. Efficacy of recombinant human granulocyte colony-stimulating factor in a murine model of pneumococcal pneumonia: effects of lung inflammation and timing of treatment. J Infect Dis. 2001; 183:70-77.
18. Allen L, Dockrell D H, Pattery T, et al. Pyocyanin Production by Pseudomonas aeruginosa Induces Neutrophil Apoptosis and Impairs Neutrophil-Mediated Host Defenses In Vivo. J Immunol. 2005; 174:3643-3649.
19. Brazil T J, Dagleish M P, McGorum B C, Dixon P M, Haslett C, Chilvers E R. Kinetics of pulmonary neutrophil recruitment and clearance in a natural and spontaneously resolving model of airway inflammation. Clin Exp Allergy. 2005; 35:854-865.
20. Walker J A, Quirke P. Viewing apoptosis through a ‘TUNEL’. J Pathol. 2001; 195:275-276.
21. Lawry J. Detection of apoptosis by the TUNEL assay. Methods Mol Med. 2004; 88:183-190.
22. Cao X, Wei G, Fang H, et al. The inositol 3-phosphatase PTEN negatively regulates Fc gamma receptor signaling, but supports Toll-like receptor 4 signaling in murine peritoneal macrophages. J Immunol. 2004; 172:4851-4857.
23. Anderson K E, Boyle K B, Davidson K, et al. CD18-dependent activation of the neutrophil NADPH oxidase during phagocytosis of Escherichia coli or Staphylococcus aureus is regulated by class III but not class I or II PI3Ks. Blood. 2008; 112:5202-5211.
24. Grunberg S M. Cyclophosphamide and etoposide for non-small cell and small cell lung cancer. Drugs. 1999; 58 Suppl 3:11-15.
25. Decker D G, Mussey E, Malkasian G D, Jr., Johnson C E. Cyclophosphamide in the treatment of ovarian cancer. Clin Obstet. Gynecol. 1968; 11:382-400.
26. van't Wout J W, et al. Effect of irradiation, cyclophosphamide, and etoposide (VP-16) on number of peripheral blood and peritoneal leukocytes in mice under normal conditions and during acute inflammatory reaction. Inflammation. 1989; 13:1-14.
27. Zuluaga A F, Salazar B E, Rodriguez C A, Zapata A X, Agudelo M, Vesga O. Neutropenia induced in outbred mice by a simplified low-dose cyclophosphamide regimen: characterization and applicability to diverse experimental models of infectious diseases. BMC Infect Dis. 2006; 6:55.
28. Cote C K, Van Rooijen N, Welkos S L. Roles of macrophages and neutrophils in the early host response to Bacillus anthracis spores in a mouse model of infection. Infect Immun. 2006; 74:469-480.
29. Mizgerd J P. Acute Lower Respiratory Tract Infection. N Engl J. Med. 2008; 358.
30. Jeyaseelan S, Young S K, Fessler M B, et al. Toll/IL-1 receptor domain-containing adaptor inducing IFN-beta (TRIF)-mediated signaling contributes to innate immune responses in the lung during Escherichia coli pneumonia. J. Immunol. 2007; 178:3153-3160.
31. Laskin D L, Weinberger B, Laskin J D. Functional heterogeneity in liver and lung macrophages. J Leukoc Biol. 2001; 70:163-170.
32. Laroux F S, Romero X, Wetzler L, Engel P, Terhorst C. Cutting edge: MyD88 controls phagocyte NADPH oxidase function and killing of gram-negative bacteria. J. Immunol. 2005; 175:5596-5600.
33. Knapp S, Leemans J C, Florquin S, et al. Alveolar macrophages have a protective antiinflammatory role during murine pneumococcal pneumonia. Am J Respir Crit Care Med. 2003; 167:171-179.
34. Zhang P, Summer W R, Bagby G J, Nelson S. Innate immunity and pulmonary host defense. Immunol Rev. 2000; 173:39-51.
35. Twigg H L, 3rd. Pulmonary host defenses. J Thorac Imaging. 1998; 13:221-233.
36. Geissmann F, Jung S, Littman D R. Blood Monocytes Consist of Two Principal Subets with Distinct Migratory Properties. Immunity. 2003; 19:71.
37. van Rijt L S, Kuipers H, Vos N, Hijdra D, Hoogsteden H C, Lambrecht B N. A rapid flow cytometric method for determining the cellular composition of bronchoalveolar lavage fluid cells in mouse models of asthma. J Immunol Methods. 2004; 288:111-121.
38. von Garnier C, Filgueira L, Wikstrom M, et al. Anatomical location determines the distribution and function of dendritic cells and other APCs in the respiratory tract. J Immunol. 2005; 175:1609-1618.
39. Vermaelen K, Pauwels R. Accurate and simple discrimination of mouse pulmonary dendritic cell and macrophage populations by flow cytometry: methodology and new insights. Cytometry A. 2004; 61:170-177.
40. GeurtsvanKessel C H, Willart M A, van Rijt L S, et al. Clearance of influenza virus from the lung depends on migratory langerin+CD11b− but not plasmacytoid dendritic cells. J Exp Med. 2008; 205:1621-1634.
41. Soehnlein O, Zemecke A, Eriksson E E, et al. Neutrophil secretion products pave the way for inflammatory monocytes. Blood. 2008; 112:1461-1471.
42. Maus U A, Backi M, Winter C, et al. Importance of phosphoinositide 3-kinase gamma in the host defense against pneumococcal infection. Am J Respir Crit. Care Med. 2007; 175:958-966.
43. McColl S R, Clark-Lewis I. Inhibition of murine neutrophil recruitment in vivo by CXC chemokine receptor antagonists. J Immunol. 1999; 163:2829-2835.
44. Mehrad B, Strieter R M, Moore T A, Tsai W C, Lira S A, Standiford T J. CXC chemokine receptor-2 ligands are necessary components of neutrophil-mediated host defense in invasive pulmonary aspergillosis. J Immunol. 1999; 163:6086-6094.
45. Tsai W C, Strieter R M, Mehrad B, Newstead M W, Zeng X, Standiford T J. CXC chemokine receptor CXCR2 is essential for protective innate host response in murine Pseudomonas aeruginosa pneumonia. Infect Immun. 2000; 68:4289-4296.
46. Tateda K, Moore T A, Newstead M W, et al. Chemokine-dependent neutrophil recruitment in a murine model of Legionella pneumonia: potential role of neutrophils as immunoregulatory cells. Infect Immun. 2001; 69:2017-2024.
47. Introna M, Bast R C, Jr., Tannenbaum C S, Hamilton T A, Adams D O. The effect of LPS on expression of the early “competence” genes JE and KC in murine peritoneal macrophages. J Immunol. 1987; 138:3891-3896.
48. Rossi D L, Hurst S D, Xu Y, et al. Lungkine, a novel CXC chemokine, specifically expressed by lung bronchoepithelial cells. J Immunol. 1999; 162:5490-5497.
49. Smith J B, Herschman H R. Glucocorticoid-attenuated response genes encode intercellular mediators, including a new C—X—C chemokine. J Biol Chem. 1995; 270:16756-16765.
50. Wolpe S D, Sherry B, Juers D, Davatelis G, Yurt R W, Cerami A. Identification and characterization of macrophage inflammatory protein 2. Proc Natl Acad Sci USA. 1989; 86:612-616.
51. Jones M R, Simms B T, Lupa M M, Kogan M S, Mizgerd J P. Lung NF-kappaB activation and neutrophil recruitment require IL-1 and TNF receptor signaling during pneumococcal pneumonia. J Immunol. 2005; 175:7530-7535.
52. Mizgerd J P, Lupa M M, Hjoberg J, et al. Roles for early response cytokines during Escherichia coli pneumonia revealed by mice with combined deficiencies of all signaling receptors for TNF and IL-1. Am J Physiol Lung Cell Mol Physiol. 2004; 286:L1302-1310.
53. Mizgerd J P, Spieker M R, Doerschuk C M. Early response cytokines and innate immunity: essential roles for TNF receptor 1 and type I IL-1 receptor during Escherichia coli pneumonia in mice. J Immunol. 2001; 166:4042-4048.
54. Mehrad B, Standiford T J. Role of cytokines in pulmonary antimicrobial host defense. Immunol Res. 1999; 20:15-27.
55. Nuckton T J, Alonso J A, Kallet R H, et al. Pulmonary dead-space fraction as a risk factor for death in the acute respiratory distress syndrome. N Engl J Med. 2002; 346:1281-1286.
56. Heit B, Robbins S M, Downey C M, et al. PTEN functions to ‘prioritize’ chemotactic cues and prevent ‘distraction’ in migrating neutrophils. Nat Immunol. 2008; 9:743-752.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims

What is claimed is:

1. A method of treating a patient for neutropenia-associated pneumonia, comprising the step of:
(a) administering to the patient in need thereof a therapeutically effective amount of a pharmaceutically-acceptable, small-molecule PTEN inhibitor.

2. The method of claim 1 wherein the patient is diagnosed to have neutropenia-associated pneumonia.

3. The method of claim 1 further comprising the step of diagnosing the neutropenia-associated pneumonia.

4. The method of claim 1 wherein the patient is undergoing or to undergo anti-cancer chemotherapy or radiotherapy.

5. The method of claim 1, further comprising the step of treating the patient with broad-spectrum antibiotic therapy or granulocyte colony-stimulating factor (G-CSF) therapy.

6. The method of claim 1 further comprising the step of detecting a resultant alleviation of the neutropenia-associated pneumonia.

7. The method of claim 1, wherein the PTEN inhibitor is (I) an ascorbic acid-based PTEN inhibitor.

8. The method of claim 1, wherein the PTEN inhibitor is (II) a 1,2,3-triazole PTEN inhibitor.

9. The method of claim 1, wherein the PTEN inhibitor is (III) a diamide PTEN inhibitor.

10. The method of claim 1, wherein the PTEN inhibitor is (IV) an aryl imidazole carbonyl PTEN inhibitor.

11. The method of claim 1, wherein the PTEN inhibitor is (V) a polyamide PTEN inhibitor.

12. The method of claim 1, wherein the PTEN inhibitor is (VI) a commercial PTEN inhibitor selected from the group consisting of:
(a) Deltamethrin; (S)-a-cyano-3-phenoxybenzyl(1R)-cis-3-(2,2 dibromovinyl)-2,2-dimethylcyclopropanecarboxylate;
(b) Alendronate, sodium, trihydrate;
(c) N-(9,10-Dioxo-9,10-dihydrophenanthren-2-yl)-2,2-dimethylpropionamide;
(d) 5-benzyl-3furylmethyl (1R,S)-cis,trans-chrysanthemate;
(e) Suramin, Sodium Salt; 8,8′-[carbonylbis[imino-3,1phenylenecarbonylimino (4-methyl-3,1-phenylene)carbonylimino]]bis-, hexasodium salt;
(f) 4-methoxyphenacyl bromide;
(g) 1,4-dimethylendothall; 1,4-dimethyl-7-oxabicyclo[2.2.1]heptane-2,3-dicarboxylic acid;
(h) Cantharidic acid; 2,3-dimethyl-7-oxabicyclo[2.2.1]heptane-2,3dicarboxylic acid;
(i) Sodium Stibogluconate; antimony sodium gluconate;
(j) 3,4-Dephostatin, ethyl-;
(k) Fenvalerate; α-cyano-3-phenoxybenzyl-α-(4-chlorophenyl)isovalerate;
(l) α-naphthyl acid phosphate, monosodium salt;
(m) β-glycerophosphate, disodium salt, pentahydrate;
(n) Endothall; 7-oxabicyclo[2.2.1]heptane-2,3dicarboxylic acid; and
(o) Cypermethrin; (R,S)-α-cyano-3-phenoxybenzyl-3(2,2-dichlorovinyl)-2,2-dimethylcyclopropanecarboxylate; (1R)-(R)cyano(3-phenoxyphenyl)methyl 3-(2,2-dichlorovinyl)-2,2-dimethylcyclopropanecarboxylate.

13. The method of claim 1, wherein the PTEN inhibitor is (VII) a 1,10-phenanthroline-5,6-dione PTEN inhibitor.

14. The method of claim 1, wherein the PTEN inhibitor is (VIII) a substituted phenathrene-9-10-dione PTEN inhibitor.

15. The method of claim 1, wherein the PTEN inhibitor is (IX) an Isatin (1H-indole-2,3-dione) PTEN inhibitor.

16. The method of claim 1, wherein the PTEN inhibitor is (X) a substituted phenanthren-9-ol PTEN inhibitor.

17. The method of claim 1, wherein the PTEN inhibitor is (XI) a substituted naphthalene-1,2-dione PTEN inhibitor.

18. The method of claim 1, wherein the PTEN inhibitor is (XII) a substituted naphthalene-1,4-dione PTEN inhibitor.

19. The method of claim 1, wherein the PTEN inhibitor is (XIII) a vanadate-based PTEN inhibitor.

20. The method of claim 1, wherein the PTEN inhibitor is (XIV) a T1-loop binding element containing PTEN inhibitor.

21. The method of claim 1 wherein the PTEN inhibitor is administered in conjunction with, and optionally coformulated with, a therapeutically-effective amount of a broad-spectrum antibiotic or granulocyte colony-stimulating factor (G-CSF).

22. A composition adapted to treating a patient for neutropenia-associated pneumonia, comprising a therapeutically-effective amount of a small-molecule PTEN inhibitor copackaged or coformulated in unit dosages with a broad-spectrum antibiotic or granulocyte colony-stimulating factor (G-CSF).
